(19) United States
(12) Patent Application Publication
(10) Pub. No.: US 2006/0264386 A1
(43) Pub. Date: Nov. 23, 2006

(54) Pharmaceutical formulations of 6-11 bicyclic ketolide derivatives and related macrolides and methods for preparation thereof

(76) Inventors: Rongqi Sun, Highland Park, NJ (US); Michelle Wang, Newton, MA (US); Yat Sun Or, Watertown, MA (US); Zhe Wang, Hockesin, DE (US)

Correspondence Address: ELMORE PATENT LAW GROUP, PC, 209 MAIN STREET, N. CHELMSFORD, MA, 01863, US

(21) Appl. No.: 11/435,153

(22) Filed: May 16, 2006

Related U.S. Application Data
(60) Provisional application No. 60/681,702, filed on May 17, 2005.

(51) Int. Cl. A61K 31/7052

(52) U.S. Cl. 514/28

(57) ABSTRACT

The present invention provides pharmaceutical compositions and solid formulations comprising EP-013420 of form I, form II, form Ia, amorphous form and monohydrate and the methods of formulation preparations. The present invention provides methods of treating bacterial infections by administering the pharmaceutical compositions to a subject in need of such treatment.

### RELATED APPLICATION

This application claims the benefit of U.S. Provisional Application No. 60/681,702, filed on May 17, 2005. The entire teachings of the above application are incorporated herein by reference.

### TECHNICAL FIELD

The present invention related to stable tablet, bead, and granulate formulations of 6-11 bicyclic ketolide derivative known as EP-13420. The present invention also relates to methods for the preparation of these formulations.

### BACKGROUND OF THE INVENTION

Macrolide antibiotics play a therapeutically important role, particularly with the emergence of new pathogens. Structural differences are related to the size of the lactone ring and to the number and nature (neutral or basic) of the sugars. Macrolides are classified according to the size of the lactone ring (12, 14, 15 or 16 atoms). The macrolide antibiotic family (14-, 15- and 16-membered ring derivatives) shows a wide range of characteristics (antibacterial spectrum, side-effects and bioavailability). Among the commonly used macrolides are erythromycin, clarithromycin, and EP-013420. Macrolides possessing a 3-oxo moiety in place of the 3-cladinose sugar are known as ketolides and have shown enhanced activity towards gram-negative bacteria and macrolide resistant gram-positive bacteria. The search for macrolide compounds which are active against MLSB-resistant strains (MLS_{B}=Macrolides-Lincosamides-type B Streptogramines) has become a major goal, together with retaining the overall profile of the macrolides in terms of stability, tolerance and pharmacokinetics.

In general, it is known that the absorption and bioavailability of any particular therapeutic agent, with a chosen polymorphic form, can be affected by numerous factors when dosed orally.

The following non-comprehensive listing can be factors involved in drug bioavailability.

(1) The particular dosage form can affect bioavailability. For example, the gastric residence time of a tablet or capsule can be significantly longer than that of a suspension, and the difference may vary depending on whether the subject has eaten or is fasted.

(2) The pH of the stomach varies, between the fed and fasted state, with the amount of food therein, and drugs which are decomposition-sensitive to pH can be affected accordingly.

(3) The capacity of the liver to metabolize an absorbed drug (so-called “first pass” metabolism) may vary with the type of meal eaten. For example some vegetables (such as brussels sprouts) can stimulate first pass metabolism of some drugs, but not others. Grapefruit juice, on the other hand, may inhibit first pass metabolism of some drugs.

(4) Bile, which is released from the gall bladder into the small intestine when a meal is ingested, has the ability to solubilize poorly-soluble drugs and thus increase bioavailability.

(5) The presence of food in the gastrointestinal (GI) tract. In general, the gastric residence time of a drug is usually significantly longer in the presence of food than in the fasted state. If the bioavailability of a drug is affected beyond a certain point due to the presence of food in the GI tract, the drug is said to exhibit a “food effect”. Food effects are important inasmuch as, when a drug exhibits an adverse food effect, there is risk associated with administering it to a patient who has eaten recently. The risk derives from the potential that absorption into the bloodstream may be adversely affected to the point that the patient risks insufficient absorption to remediate the condition for which the drug was administered.

Additional factors can also be involved in the absorption and bioavailability of a particular drug, and absorption can actually be increased as well as decreased. These additional factors include, for example, pH-dependent solubility, site-specific intestinal permeation rate, instability to intestinal enzymes, susceptibility to first pass metabolism, and instability to colonic bacteria. Given the plethora of factors which can influence bioavailability, there usually is no way to predict, in the absence of actual testing, whether a particular drug will exhibit a food effect. For example, Toothaker and Welling, Ann. Rev. Pharmacol. Toxicol., 1980, 173-99, discuss various drugs whose absorption is delayed in the presence of food (cephalexin, cefaclor, metronidazole, aspirin, alclofenac, indoprofen, digoxin, cimetidine), whose absorption may be unaffected by food (ampicillin, erythromycin estolate, spiramycin, propylthiouracil, oxazepam, bendroflumethiazide), and whose absorption is increased in the presence of food (erythromycin ethylsuccinate, nitrofurantoin, 8-methoxsalen, propranolol, metoprolol, dicoumarol, diazepam, hydrochlorothiazide).

### SUMMARY OF THE INVENTION

The present invention related to stable pharmaceutical formulations and to methods for the preparation of these formulations of EP-13420 which has the following formula:

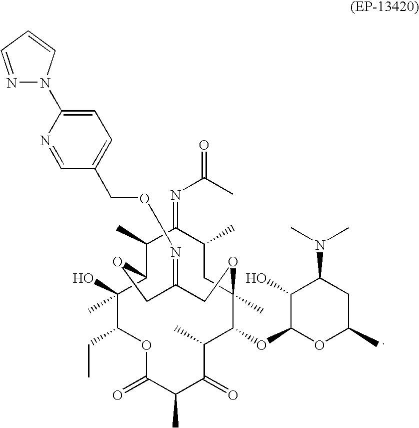

### DESCRIPTION OF THE INVENTION

This invention provides oral dosage form of crystalline form I, form Ia, form II, amorphous form or monohydrate EP-013420 (Provisional Application 60/546,433), which can be administered to a mammal (including humans). The dosage form comprises EDP-013420 and, pharmaceutically acceptable carrier, as hereinafter further detailed and described. The dosage form is in the form of tablets, beads or granulates.

The core tablets, beads or granulates may be coated to provide improved swallowability, moisture and light protection, gastric pH-resistance, covering taste-bitterness and better appearance.

In a further aspect, this invention provides a method for treating a microbial infection in a mammal which comprises administering, to a mammal that has eaten in need of such treatment, an antimicrobially effective amount of EDP-013420 in an oral dosage form which exhibits substantially increased stability of drug product.

In a further aspect, this invention provides a therapeutic package suitable for clinical studies and commercial sale, comprising a container, an oral dosage form of EP-013420 which does not exhibit an adverse food effect contained therein, and, associated with said container, written matter non-limited as to whether the dosage form can be taken with or without food.

For purposes of this invention EP-013420 may be administered alone or in combination with other therapeutic agents.

It is generally assumed and observed that the in vitro dissolution rate of dosage forms exhibits a rank order correlation with in vivo dissolution, particularly for a single dosage form type, e.g. tablets, which vary systematically in composition. Thus in vitro dissolution evaluation serves an important role in control of the quality of manufactured dosage forms. It is not necessarily true that the in vitro dissolution rate is exactly the same as the in vivo dissolution rate. This is not surprising, since the artificial conditions of an in vitro dissolution test (e.g. vessel geometry, stirring rate, stirring method, and so forth) are not identical to the conditions under which a dosage form disintegrates and dissolves in the GI tract.

When comparing dosage forms of different type, e.g. capsules and tablets, in vitro dissolution rate should correlate roughly with in vivo dissolution rate. However, subtle differences exist between the disintegration mechanisms of capsules and tablets. For capsules, at least partial dissolution of the gelatin shell must precede complete dissolution of the enclosed drug. Furthermore, capsule shells generally dissolve first at the capsule ends, and later at the capsule center. Tablets, on the other hand, disintegrate homogeneously. Thus subtle differences may exist in the in vitro/in vivo dissolution correlation when comparing capsules and tablets. For example, capsules and tablets which exhibit similar in vitro dissolution rates may exhibit subtle differences in in vivo dissolution rate. While such subtle differences may have no therapeutically significant effect on systemic bioavailability of an orally dosed drug, there are situations in which a significant effect may occur. For example, if a drug has the potential to exhibit an adverse food effect, drug-containing capsules and tablets which exhibit similar in vitro dissolution rates may actually differ with respect to whether an adverse food effect is observed when the dosage forms are orally dosed.

Tablets according to the invention contain, as necessary ingredients, EP-013420 and a disintegrant. Examples of tablet disintegrants are starch, pregelatinized starch, sodium starch glycolate, sodium carboxymethylcellulose, crosslinked sodium carboxymethylcellulose (sodium croscarmellose; crosslinked starch available under the registered trademark Ac-Di-Sol from FMC Corp., Philadelphia, Pa.), clays (e.g. magnesium aluminum silicate), microcrystalline cellulose (of the type available under the registered trademark Avicel from FMC Corp. or the registered trademark Emcocel from JRS Pharma, Patterson, N.Y.), alginates, gums, surfactants, effervescent mixtures, hydrous aluminum silicate, cross-linked polyvinylpyrrolidone (available commercially under the registered trademark PVP-XL from International Specialty Products, Inc.), and others as known in the art. Preferred disintegrants for EP-013420 tablets are sodium croscarmellose (Ac-Di-Sol), sodium starch glycolate (available commercially under the registered trademarks Primojel from Avebe (Union, N.J.) or Generichem, (Little Falls, N.J.) and Explotab from JRS Pharma), microcrystalline cellulose (Avicel), and cross-linked polyvinylpyrrolidone (PVP-XL). EP-013420 tablets of this invention comprise EP-013420 and 1-25% disintegrant, preferably 3-15% disintegrant based on total tablet weight. For example, a 463.5 mg tablet (250 mg activity EP-013420) may contain 9 mg sodium croscarmellose and 27 mg pregelatinized starch.

In addition to the active ingredient EP-013420 and a disintegrant, tablets according to this invention may be formulated to optionally include a variety of conventional excipients, depending on the exact formulation, such as binders, flavorings, buffers, diluents, colors, lubricants, sweetening agents, thickening agents, and glidants. Some excipients can serve multiple functions, for example as both binder and disintegrant.

Examples of binders are acacia, cellulose derivatives (such as methylcellulose and carboxymethylcellulose, hydroxypropylmethylcellulose, hydroxypropylcellulose, hydroxyethylcellulose), gelatin, glucose, dextrose, xylitol, polymethacrylates, polyvinylpyrrolidone, starch paste, sucrose, sorbitol, pregelatinized starch, gum tragacanth, alginic acids and salts thereof such as sodium alginate, magnesium aluminum silicate, polyethylene glycol, guar gum, bentonites, and the like. A preferred binder for EP-013420 tablets is pregelatinized starch (available, for example, under the registered trademark Starch 1500, from Colorcon, Inc., West Point, Pa.).

Flavors incorporated in the composition may be chosen from synthetic flavor oils and flavoring aromatics and/or natural oils, extracts from plants leaves, flowers, fruits, and so forth and combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth. The amount of flavoring may depend on a number of factors including the organoleptic effect desired. Generally the flavoring will be present in an amount of from 0.5 to about 3.0 percent by weight based on the total tablet weight, when a flavor is used.

A variety of materials may be used as fillers or diluents. Examples are spray-dried monohydrate or anhydrous lactose, sucrose, dextrose, mannitol, sorbitol, starch (e.g. starch 1500), cellulose (e.g. microcrystalline cellulose; Avicel), dihydrated or anhydrous dibasic calcium phosphate (available commercially under the registered trademark Emcompress from JRS Pharma or A-Tab and Di-Tab from Rhone-Poulenc, Inc., Monmouth Junction, N.J.), calcium carbonate, calcium sulfate, and others as known in the art.

Lubricants can also be employed herein in the manufacture of certain dosage forms, and will usually be employed when producing tablets. Examples of lubricants are magnesium stearate, stearic acid, glycerylbehaptate, polyethylene glycol, ethylene oxide polymers (for example, available under the registered trademark Carbowax from Union Carbide, Inc., Danbury, Conn.), sodium lauryl sulfate, magnesium lauryl sulfate, sodium oleate, sodium stearyl fumarate, DL-leucine, colloidal silica, and others as known in the art. Preferred lubricants are magnesium stearate, and mixtures of magnesium stearate with sodium lauryl sulfate. Lubricants generally comprise 0.5 to 7.0% of the total tablet weight.

Other excipients such as glidants and coloring agents may also be added to EP-013420 tablets. Coloring agents may include titanium dioxide and/or dyes suitable for food such as those known as F. D. & C, dyes and natural coloring agents such as grape skin extract, beet red powder, beta carotene, annato, carmine, turmeric, paprika, and so forth. A coloring agent is an optional ingredient in the compositions of this invention, but when used will generally be present in an amount up to about 3.5 percent based on the total tablet weight.

As known in the art, tablet blends may be dry-granulated or wet-granulated before tableting. Alternatively, tablet blends may be directly compressed. The choice of processing approach depends upon the properties of the drug and chosen excipients, for example particle size, blending compatibility, density and flowability. For EP-013420 tablets, granulation is preferred, with wet granulation being most preferred. EP-013420 may be wet-granulated, and then other excipients may be added extragranularly. Alternatively, EP-013420 and one or more excipients may be wet-granulated. In addition, tablets may also be coated, with a coating that exhibits little or no effect on or interference with tablet dissolution, to assure ease of swallowing or to provide an elegant appearance.

In a preferred embodiment, tablets of this invention are film-coated to provide improved stability and acid stability, ease of swallowing and an elegant appearance. Many polymeric film-coating materials are known in the art. A preferred film-coating material is hydroxypropylmethylcellulose (HPMC). HPMC may be obtained commercially, for example from Colorcon Corp., in coating formulations containing excipients which serve as coating aids, under the registered trademark Opadry. Opadry formulations may contain lactose, polydextrose, triacetin, polyethyleneglycol, polysorbate 80, titanium dioxide, and one or more dyes or lakes. Other suitable film-forming polymers also may be used herein, including, hydroxypropylcellulose, and acrylate-methacrylate copolymers.

The tableting process itself is otherwise standard and readily practiced by forming a tablet from a desired blend or mixture of ingredients into the appropriate shape using a conventional tablet press. Tablet formulation and conventional processing techniques have been widely described, for Example in Pharmaceutical Dosage Forms: Tablets; Edited By Lieberman, Lachman, and Schwartz; Published by Marcel Dekker, Inc., 2d Edition, Copyright 1989, the text of which is herein incorporated by reference.

The EP-013420 dosage forms of this invention also include powders to make oral suspensions, and also the oral suspensions themselves. Generally the powder is a non-caking, free flowing powder which is sold direct to pharmacies or other retail outlets and then made up into the actual suspension by a pharmacist. The oral suspension is thus the actual dosage form ingested by patients. The typical shelf life for a suspension is about five days because EP-013420 therapy is generally of three to seven days duration.

EP-013420 suspensions according to the invention contain, as necessary ingredients in addition to EP-013420, one or more thickening agents in a total amount of 0.1 to 2%, and a buffer or pH-altering agent in an amount of 0.1 to 2.5%, with percentages being based on the weight of the dry powder formulation. Dispersing agents may also be used in an amount of from 0.05 to 2%. Preservatives may also be used in an amount from 0.1 to 2%.

Suitable thickening agents function as suspending agents and include, for example, hydrocolloid gums known for such purpose, examples of which include xanthan gum, guar gum, locust bean gum, gum tragacanth, and the like. Alternatively, synthetic suspending agents may be used such as sodium carboxymethylcellulose, polyvinylpyrrolidone, hydroxypropylcellulose and the like.

Dispersing agents include colloidal silicon dioxide, available from Cabot Corporation, Boston, Mass. under the trade designation Cab-O-Sil.

For the purpose of preparing formulations of a powder for oral suspension, the bitter taste of EP-013420 may be masked by sweeteners, such as sugar or Sorbitol in drinking water; and by including a buffer or pH-altering agent which will provide an appropriate pH in the constituted suspension. Maintenance of a chosen pH minimizes the quantity of EP-013420 in solution, and thus masks the bitter taste of the drug. Many combinations of flavors or flavor systems may be used in addition to mask the bitter taste of EP-013420. Preferred flavors are those which provide a constant flavor for approximately three to seven days at the neutral pH of the formulation after constitution. A preferred flavor system consists of spray dried cherry # 11929, artificial creme de vanilla # 11489, and spray-dried artificial banana # 15223 available commercially from Bush Boake Allen, Inc., Chicago, Ill. Artificial sweeteners may also be used.

A powder used to make a suspension herein may also contain conventional optional ingredients such as (1) wetting agents such as sorbitan monolaurate, polysorbate 80, and sodium lauryl sulfate; (2) anti-foaming agents and (3) sweeteners and fillers such as glucose. The powder may also contain a buffer to maintain a high pH upon reconstitution, as discussed above. Suitable buffers and pH-altering agents include anhydrous tribasic sodium phosphate, anhydrous sodium carbonate, glycine, and the like. Suitable preservatives are well known, for example sodium benzoate and the like. After swallowing, EP-013420 from a suspension dissolves quickly.

In the preparation of EP-013420 powder for oral suspension formulations, all ingredients may be blended together and deagglomerated, as known in the art. Preferably, EP-013420 and flavors are blended, and other ingredients are separately blended. Finally, these two blends are blended and deagglomerated.

Preferred oral suspensions are those which resuspend easily after constitution with aqueous media and which do not cake on storage after constitution. Preferred suspensions contain sucrose NF, when sucrose is used, and anhydrous excipients when available, to assure facile suspension upon constitution. The drug-containing powder is generally reconstituted with water.

Suspensions of this invention exhibit about 90% dissolution of EP-013420 in vitro in about 15 minutes.

Definitions

Listed below are definitions of various terms used to describe this invention. These definitions apply to the terms as they are used throughout this specification and claims, unless otherwise limited in specific instances, either individually or as part of a larger group.

As used herein, “substantially pure” means a compound having a purity of at least 90 percent, preferably at least 95 percent, and more preferably at least 98 percent.

The term “active ingredient” or “active ingredients,” as used herein, refers to any of the polymorphic forms, or combinations thereof, delineated herein (i.e. Form I, Form II, Form Ia, or amorphous EP-13420, or combinations thereof, or combinations with other polymorphic forms of EP-13420). Preferably, Form I or Form II EP-13420 in their pure forms is used in the pharmaceutical compositions of the present invention.

The term “subject” as used herein refers to an animal. Preferably the animal is a mammal. More preferably the mammal is a human. A subject also refers to, for example, dogs, cats, horses, cows, pigs, guinea pigs, fish, birds and the like.

By a “therapeutically effective amount” of an active ingredient or ingredients of the present invention is meant an amount of the active ingredient(s) which confer(s) a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the active ingredient(s) described herein may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific active ingredient employed; and like factors well known in the medical arts.

According to the methods of treatment of the present invention, bacterial infections, cystic fibrosis, and inflammatory conditions are treated or prevented in a subject such as a human or another animal by administering to the patient a therapeutically effective amount of an active ingredient or ingredients of the present invention, in such amounts and for such time as is necessary to achieve the desired result.

As used herein, unless otherwise indicated, the term “bacterial infection(s)” or “protozoa infections” includes, but is not limited to, bacterial infections and protozoa infections that occur in mammals, fish and birds as well as disorders related to bacterial infections and protozoa infections that may be treated or prevented by administering antibiotics such as polymorphic forms of the present invention. Such bacterial infections and protozoa infections and disorders related to such infections include, but are not limited to, the following: pneumonia, otitis media, sinusitus, bronchitis, tonsillitis, cystic fibrosis (CF) and mastoiditis, related to infection by Streptococcus pneumoniae, Haemophilus influenzae, Moraxella catarrhalis, Staphylococcus aureus, Peptostreptococcus spp., or Pseudomonas spp.; pharynigitis, rheumatic fever, and glomerulonephritis, related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S. and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp. odontogenic infection related to infection by viridans streptococci; persistent cough related to infection by Bordetella pertussis; gas gangrene related to infection by Clostridium perfringens or Bacteroides spp.; Skin infection by S. aureus, Propionibacterium acne; atherosclerosis related to infection by Helicobacter pylori or Chlamydia pneumoniae; or the like.

Bacterial infections and protozoa infections and disorders related to such infections that may be treated or prevented in animals include, but are not limited to, the following: bovine respiratory disease related to infection by P. haemolytica., P. multocida, Mycoplasma bovis, or Bordetella spp.; cow enteric disease related to infection by E. coli or protozoa (i.e., coccidia, cryptosporidia, etc.), dairy cow mastitis related to infection by S. aureus, S. uberis, S. agalactiae, S. dysgalactiae, Klebsiella spp., Corynebacterium, or Enterococcus spp.; swine respiratory disease related to infection by A. pleuropneumoniae., P. multocida, or Mycoplasma spp.; swine enteric disease related to infection by E. coli, Lawsonia intracellularis, Salmonella spp., or Serpulina hyodyisinteriae; cow footrot related to infection by Fusobacterium spp.; cow metritis related to infection by E. coli; cow hairy warts related to Infection by Fusobacterium necrophorum or Bacteroides nodosus; cow pink-eye related to infection by Moraxella bovis, cow premature abortion related to infection by protozoa (i.e. neosporium); urinary tract infection in dogs and cats related to infection by E. coli; skin and soft tissue infections in dogs and cats related to infection by S. epidermidis, S. intermedius, coagulase neg. Staphylococcus or P. multocida; and dental or mouth infections in dogs and oats related to infection by Alcaligenes spp., Bacteroides spp., Clostridium spp., Enterobacter spp., Eubacterium spp., Peptostreptococcus spp., Porphfyromonas spp., Campylobacter spp., Actinomyces spp., Erysipelothrix spp., Rhodococcus spp., Trypanosoma spp., Plasmodium spp., Babesia spp., Toxoplasma spp., Pneumocystis spp., Leishmania spp., and Trichomonas spp. or Prevotella spp. Other bacterial infections and protozoa infections and disorders related to such infections that may be treated or prevented in accord with the method of the present invention are referred to in J. P. Sanford at al., “The Sanford Guide To Antimicrobial Therapy,” 26th Edition, (Antimicrobial Therapy, Inc., 1996).

The invention further provides compositions and methods of treating patients suffering from an inflammatory condition comprising administering to a patient in need thereof, a therapeutically effective amount of at least one compound of the invention. Specific examples of inflammatory conditions treatable according to the invention include, but are not limited to, scleritis; epi-scleritis; allergic conjunctivitis; pulmonary inflammatory diseases, particularly cystic fibrosis (CF), asthma, chronic obstructive pulmonary disease (COPD), allergic bronchopulmonary aspergillosis (ABPA), and sarcoidosis; procto-sigmoiditis; allergic rhinitis; arthritis; tendonitis; apthous stomatitis; and inflammatory bowel disease.

The invention further provides compositions and methods for i) prophylactic treatment of those patients susceptible to the symptoms CF including pulmonary infection and inflammation associated with CF, ii) treatment at the initial onset of symptoms of pulmonary infection and inflammation associated with CF, and iii) treatment of ongoing or relapsing symptoms of infection and inflammation associated with CF. In accordance with the invention a compound of the invention is administered to a patient in need of treatment for CF, in amount sufficient to prevent, diminish or eradicate symptoms of CF including chronic pulmonary inflammation and infection.

Pharmaceutical Compositions

Pharmaceutical compositions of the present invention comprise a therapeutically effective amount of any one of the active ingredients of the present invention, or a combination thereof, optionally formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In one embodiment a preferred suspension formulation of the invention comprises 60-1600 mg of amorphous EP-13420 in a simple syrup:sterile water (4:56, v:v) solution.

In another embodiment, suspensions are made that advantageously have the taste of the pharmaceutical ingredients masked such as that described in US Pat. Pub. No: 2004/0142029 incorporated herein by reference. One pharmaceutical formulation of EP 13420 (Form I, Form Ia, Form II, monohydrate, amorphous, or any combination thereof) having a masked taste by sucrose, preferably in the form of a suspension in an aqueous vehicle, comprises: water and Simple Syrup.

In another taste masking embodiment, the formulation comprises from about 15 to about 30% of EP 13420 in any form or any combination of forms mixed with from about 60% to about 80% of an ester of glycerol or of a fatty acid, to which a wax is optionally added, and to which a surfactant is added, and wherein the composition is prepared by a spray-cooling process which can produce a particle size of less than about 350 microns. The esters of glycerol or of fatty acid used in this embodiment have the following characteristics: melting temperature in the range of from about 25.degree. C. to about 100.degree. C., preferably from about 25.degree. C. to about 70.degree. C. and stability in the molten state. The ester of glycerol may be chosen from glyceryl stearate or glyceryl palmitostearate, in particular Precirol.®. The ester of glycerol is advantageously between 50 and 85% by weight of the total mixture of the composition; it is preferably between 60 and 80% by weight, and more particularly between 70 and 80% by weight. The wax which can be optionally added may advantageously be carnauba wax, or it may also be chosen from paraffin or beeswax or candelilla wax. When a wax is added to the composition, it may be added in a proportion of from about 4% to about 10% by weight of the total mixture of the composition and in a ratio of from about 5% to about 20% with respect to the ester of glycerol introduced. When a fatty acid is introduced into the composition, this fatty acid is advantageously chosen from palmitic, myristic or stearic acid. The fatty acid is introduced in a proportion of from about 60% to about 80% by weight of the total mixture of the composition. The surfactant introduced into the composition is advantageously chosen from lecithins, in particular soybean lecithin, or surfactants of the family of sorbitan esters having an HLB of less than 7. The surfactant is added in a proportion of from about 1% to about 3% by weight of the total mixture of the composition.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include, tablets, beads, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, crospovidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In another embodiment, the invention provides a tablet comprising an alginate matrix consisting of a water soluble alginate salt and a complex salt of alginic acid, active ingredient that is any form of EP 13420 described herein or any combination thereof, an inorganic salt capable of donating a proton and having a pKa value in water of 4.0 to 9.0. Alginate formulations are generally described in WO 2004/056344 incorporated herein by reference. An alginate matrix suitable with the invention comprises a water-soluble alginate and a complex salt of alginic acid. The water soluble alginate in the composition is typically an alkali salt of alginic acid such as a potassium or sodium salt, or a magnesium salt or an ammonium salt. A complex salt of alginic acid is typically a sodium-calcium complex salt of alginic acid. The weigh ratio of a soluble alginate to a complex salt of alginic acid may vary from about 16:1 to 1:1 preferably from about 8:1 to 2:1. The same ratio applies to the ratio of sodium alginate to sodium calcium alginate. Preferably the amount of soluble alginate in a composition varies from about 6% to about 25% of the total weight of the composition and the amount of the complex salt of alginic acid varies from about 0.5% to about 10% of the total weight of the composition. The mixture may be granulated according to conventional granulation technology and by drying the obtained granules using conventional drying technology. The dried granules may optionally be resized. In the case the composition is a capsule, the granules are filed into the capsule, (e.g. gelatin capsule. In the case the composition is a tablet, the granules may be mixed with glindants/lubricants and compressed into tablets using conventional technology.

In another embodiment, the invention provides a “fast melt” formulation. Such fast melt formulations are typically in the form of a tablet or lozenge that dissolve or disperse in a patient's mouth within a minute without the need of water or chewing. Such fast melt formulations are described in WO 03/074029, incorporated herein by reference. In certain embodiments, the formulation comprises a non-compressed, free flowing plurality of particles comprising at least one form of EP 13420 of the invention (Form I, Form Ia, Form II, monohydrate, amorphous or any combination thereof) and a water-soluble excipient, the particles having a mean diameter of greater than 10 microns to about 1 mm, the particles comprising at least about 50% active ingredient and the formulation dissolving in the patients mouth within 1 minute after administration without the co-administration of fluid. The water soluble excipient of the formulation can be a sugar alcohol including, but not limited to sorbitol, manitol, maltitol, reduced starch saccharide, xylitol, reduced parationse, erythritol and combinations thereof. Other suitable water soluble excipients include gelatin, partially hydrolyzed gelatin, hydrolyzed dextran, dextrin, alginate and mixtures thereof. Salivary stimulants such as citric acid, carbonate sources and the like and sweenters such as saccharin salts, and aspartame may optionally be included.

The solid dosage forms of tablets, dragees, capsules, pills, beads and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. In one embodiment, the invention provides a formulation comprising at least one form of EP-13420 (Form I, Form Ia, Form II, monohydrate, amorphous or any combination thereof) in a buccal aersol spray comprising polar or non-polar solvents similar to that described in US Pat Pub 2003/0082107, incorporated herein by reference. In this embodiment a propellant-free buccal spray formulation for transmucosal administration comprises at least one form of EP-13420 (Form I, Form Ia, Form II, monohydrate, amorphous or any combination thereof) and a polar or non-polar solvent in an amount between 30-99%. Optionally a propellant may be used in the amount of 2-10% by weight of the total composition if a propellant buccal spray is desired.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The total daily dose of the pharmaceutical compositions of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The pharmaceutical compositions, as described herein, can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of a therapeutically effective amount of a pharmaceutical composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active ingredient (w/w). Alternatively, such preparations may contain from about 20% to about 80% active ingredient.

Lower or higher doses than those recited above may be required. Specific dosage and treatment regimens for any particular patient will depend upon a variety of factors, including the activity of the specific pharmaceutical composition employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of any one the active ingredients, or a combination thereof, of the present invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the pharmaceutical compositions of this invention comprise a combination of an active ingredient of the present invention and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with active ingredients of this invention in a single composition.

Unless otherwise defined, all technical and scientific terms used herein are accorded the meaning commonly known to one of ordinary skill in the art. All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Core Tablets or Beads:

| Ingredient | Amount |
|---|---|
| EDP-420 API | Up to 80% |
| Diluent | 0-90% |
| Binder | 0-20% |
| Disintegrant | 0-10% |
| Glidant | 0-2% |
| Lubricant | 0-2% |
| Water or Buffer for granulation | q.s. |

Diluent used in the above composition may include Microcrystalline Cellulose, Anhydrous Lactose, Monohydrate Lactose, Silicified Microcrystalline Cellulose, Dibasic Calcium Phosphate anhydrous, Dibasic Calcium Phosphate anhydrous and Mannitol.

Examples of Binder may include, starch, pregelatinized starch, povidone, hydroxypropyl cellulose, hydroxypropyl methyl cellulose and methyl cellulose.

Examples of disintegrant used in the formula may include Pregelatinized starch, Crospovidone, Sodium starch Glycolate, and Croscarmellose sodium.

Examples of glidants are colloidal silicon dioxide and talc.

Examples of Lubricant include magnesium stearate, calcium stearate, stearic acid, hydrogenated castor oil and sodium stearyl fumarate.

Process:

The tablets can be produced by using 1) dry blend process, 2) wet granulation process, or 3) top-spray granulation process.

Beads can be produced by 1) wet granulation and milling, 2) extrusion and spheronization. The beads will be encapsulated in capsules shells.

The wet granulation process can utilize a suitable buffer to prevent any in-process degradation of the active.

Coating:

The core tablets or beads may be coated to provide one or more of the following 1) swallowability, 2) moisture and light protection 3) gastric pH-resistance, 4) better appearance.

Following are examples of coating formula:

| Ingredient | Amount |
|---|---|
| Coating polymer | Up to 90% |
| Plasticizer | 0-75% |
| Glidant | 0-75% |
| Surfactant | 0-10% |
| Solvent | q.s. |

Coating polymer could be one or combination of more than one of the following materials: hydroxypropyl cellulose, ethyl cellulose, hydroxypropylmethyl cellulose (HPMC), pH-dependent methacrylate copolymers, hydroxypropylmethyl cellulose phthalate, polyvinyl acetate phathalate, cellulose acetate phthalate, cellulose acetate butyrate.

Examples of plasticizer include triethyl citrate, triethyl phthalate, tributyl phthalate, glyceryl triacetate and dibutyl sebacate.

Surfactants such as sodium lauryl sulfate and polysorbate 80 may be used.

Solvent may be one or combination of: water and/or an organic solvent such as ethanol, methanol, isopropyl alcohol, methylene chloride and acetone.

Coating Process: Film-coating for tablets, fluidized-bed wurster coating for beads.

Encapsulation of beads: The uncoated and/or coated beads can be encapsulated in gelatin, starch or HPMC capsule shells.

Packaging:

The tablets can be packaged in the following packages:

Glass bottles with or without desiccant

HDPE bottles with or without desiccant

Clear or Colored Blister packages that may be combination of PVC, PVDC, paper and Aluminum.

Pouches of Aluminum and/or paper

Antibacterial Activity

Susceptibility tests can be used to quantitatively measure the in vitro activity of an antimicrobial agent against a given bacterial isolate. Compounds are tested for in vitro antibacterial activity by a micro-dilution method. Minimal Inhibitory Concentration (MIC) is determined in 96 well microtiter plates utilizing the appropriate Mueller Hinton Broth medium (CAMHB) for the observed bacterial isolates. Antimicrobial agents are serially diluted (2-fold) in DMSO to produce a concentration range from about 64 μg/ml to about 0.03 μg/ml. The diluted compounds (2 μl/well) are then transferred into sterile, uninoculated CAMHB (0.2 mL) by use of a 96 fixed tip-pipetting station. The inoculum for each bacterial strain is standardized to 5×10⁵ CFU/mL by optical comparison to a 0.5 McFarland turbidity standard. The plates are inoculated with 10 μl/well of adjusted bacterial inoculum. The 96 well plates are covered and incubated at 35+/−2° C. for 24 hours in ambient air environment. Following incubation, plate wells are visually examined by Optical Density measurement for the presence of growth (turbidity). The lowest concentration of an antimicrobial agent at which no visible growth occurs is defined as the MIC. The active ingredients of the invention typically will demonstrate MICs in the range from about 64 μg/ml to about 0.03 μg/ml.

All in vitro testing follows the guidelines described in the Approved Standards M7-A4 protocol, published by the National Committee for Clinical Laboratory Standards (NCCLS).

Although the invention has been described with respect to various preferred embodiments, it is not intended to be limited thereto, but rather those skilled in the art will recognize that variations and modifications may be made therein which are within the spirit of the invention and the scope of the appended claims.

What is claimed:

1. An oral dosage form of EP-013420 in tablet form comprising EP-013420 and an excipient, wherein said dosage form achieves at least about 90% dissolution of EP-013420 within about 30 minutes when an amount of the dosage form equivalent to 200 mg of EP-013420 is tested as set forth in USP test <711> in a USP-2 dissolution apparatus under conditions at least as stringent as the following: 900 ml sodium phosphate buffer pH 6.0 at 37° C., with paddles turning at 100 rpm.

2. A dosage form as defined in claim 1, wherein said tablet is produced by wet granulation.

3. A dosage form as defined in claim 1, further comprising a flavoring agent.

4. An oral dosage form of EP-013420 which is in the form of a powder for oral suspension.

5. A dosage form as defined in claim 4, wherein said suspension comprises water and Simple Syrup.

6. A dosage form as defined in claim 4, further comprising a flavoring agent.

7. A dosage form as defined in claim 6, wherein said flavoring agent is a flavor system consisting of cherry, vanilla, and banana.

8. A suspension made from the powder of claim 4.

9. An oral dosage form of EP-013420 which is in the form of a bead made by wet granulation which comprises EP-013420 and an excipient.

10. An oral dosage form of EP-013420 which is in the form of a granulate.

11. A dosage form as defined in claim 1, comprising:
58.2% EP-013420 hydrate;
6.0% pregelatinized starch;
30.9% anhydrous dibasic calcium phosphate;
2.0% sodium croscarmellose; and
2.9% lubricant.

12. A dosage form as defined in claim 1, comprising:
58.2% EP-013420;
11.1% pregelatinized starch;
25.7% anhydrous dibasic calcium phosphate;
2.0% sodium croscarmellose; and
2.9% lubricant.

13. A dosage form as defined in claim 1, comprising:
58.2% EP-013420;
3.1% pregelatinized starch;
31.3% anhydrous dibasic calcium phosphate;
4.4% sodium croscarmellose; and
2.9% lubricant.

14. A dosage form as defined in claim 1, comprising:
58.2% EP-013420;
11.1% pregelatinized starch;
23.3% anhydrous dibasic calcium phosphate;
4.4% sodium croscarmellose; and
2.9% lubricant.

15. A dosage form as defined in claim 1, comprising:
58.2% EP-013420;
3.1% maize starch;
33.8% dibasic calcium phosphate, lactose, or microcrystalline cellulose;
2.0% sodium starch glycolate or crosslinked polyvinylpyrrolidone; and
2.9% lubricant.

16. A dosage form as defined in claim 1, comprising:
58.2% EP-013420;
6.0% maize starch;
30.9% dibasic calcium phosphate, lactose, or microcrystalline cellulose;
2.0% sodium starch glycolate or crosslinked polyvinylpyrrolidone; and
2.9% lubricant.

17. A dosage form as defined in claim 1, comprising:
58.2% EP-013420;
11.1% maize starch;
25.7% dibasic calcium phosphate, lactose, or microcrystalline cellulose;
2.0% sodium starch glycolate or crosslinked polyvinylpyrrolidone; and
2.9% lubricant.

18. A dosage form as defined in claim 1, comprising:
58.2% EP-013420;
3.1% maize starch;
31.3% dibasic calcium phosphate, lactose, or microcrystalline cellulose;
4.4% sodium starch glycolate or crosslinked polyvinylpyrrolidone; and
2.9% lubricant.

19. A dosage form as defined in claim 1, comprising:
58.2% EP-013420;
6.0% maize starch;
32.2% dibasic calcium phosphate, lactose, or microcrystalline cellulose;
0.7% sodium starch glycolate or crosslinked polyvinylpyrrolidone; and
2.9% lubricant.

20. A dosage form as defined in claim 1, comprising:
58.2% EP-013420;
6.0% maize starch;
28.4% dibasic calcium phosphate, lactose, or microcrystalline cellulose;
4.4% sodium starch glycolate or crosslinked polyvinylpyrrolidone; and
2.9% lubricant.

21. A dosage form as defined in claim 4, comprising:
5% EP-013420 and
5% sucrose.

22. A dosage form as defined in claim 4, comprising:
5% EP-013420 and
5% sorbitol.

23. A dosage form as defined in claim 1 made as a tablet and coated with:
1-20% cellulosic polymer.

24. A dosage form as defined in claim 1 made as a tablet and coated with:
1-20% methacrylate polymer.

25. A dosage form as defined in claim 9 made as beads or granulate and coated with:
1-20% cellulosic polymer.

26. A dosage form as defined in claim 9 made as beads or granulate and coated with:
1-20% methacrylate polymer.

27. A dosage form as defined in claim 10 made as beads or granulate and coated with:
1-20% cellulosic polymer.

28. A dosage form as defined in claim 10 made as beads or granulate and coated with:
1-20% methacrylate polymer.
